(19) United States
(12) Patent Application Publication
(10) Pub. No.: US 2024/0119704 A1
(43) Pub. Date: Apr. 11, 2024

(54) ELECTROSURGICAL GENERATOR AND METHOD OF OPERATION THEREOF

(71) Applicant: OLYMPUS WINTER & IBE GMBH, Hamburg (DE)

(72) Inventor: Jens KRÜGER, Hamburg (DE)

(73) Assignee: OLYMPUS WINTER & IBE GMBH, Hamburg (DE)

(21) Appl. No.: 18/242,845

(22) Filed: Sep. 6, 2023

Related U.S. Application Data
(60) Provisional application No. 63/413,485, filed on Oct. 5, 2022.

(51) Int. Cl. G06V 10/764; A61B 18/12; G06V 20/64

(52) U.S. Cl. CPC G06V 10/764; A61B 18/1206; G06V 20/64; A61B 2018/00702

(57) ABSTRACT

An electrosurgical generator is provided, including: at least one interface for connecting an electrosurgical instrument to the electrosurgical generator; an electrosurgical signal generation unit for supplying an electrosurgical signal to an electrosurgical instrument connected to the electrosurgical generator; a processor configured to control the electrosurgical signal generation unit; and an instrument detection unit configured to detect the type of an electrosurgical instrument connected to the electrosurgical generator; wherein the instrument detection unit comprises a camera and an image processor, the camera being configured to acquire one or more images of an electrosurgical instrument connected to the electrosurgical generator, and the image processor being configured to analyse the one or more images to detect the type of the electrosurgical instrument; and wherein the processor is configured to control the electrosurgical signal generation unit depending on the detected type of the electrosurgical instrument.

### TECHNICAL FIELD

The present disclosure relates to electrosurgical generators. More specifically, the disclosure relates to electrosurgical generators being capable of detecting the type of an electrosurgical instrument connected to the electrosurgical generator. The present disclosure further relates to methods of operating an electrosurgical generator.

### BACKGROUND

Electrosurgery is used in modern medicine for reliably achieving a number of tissue effects. Such tissue effects include, but are not limited to, cutting, coagulating, ablating, evaporating, cauterizing, and the like.

The above tissue effects are achieved through direct or indirect application of electric current to the tissue under treatment. For direct application of the current, the tissue under treatment is contacted with one or more electrodes conducting the current into and through the tissue. For indirect application of the current, the current may be conducted into and through a medium contacting the tissue, and the medium may be heated, vaporized, or turned into a plasma for achieving the desired tissue effect. In other indirect applications, the current may be used to create ultrasonic vibrations of a sonotrode contacting the tissue under treatment. In other applications, current is used to create electromagnetic waves causing a tissue effect. In most cases, electrosurgery uses high frequency alternating currents, commonly referred to as electrosurgical signals or electrosurgical therapy signals.

Generally, electrosurgical applications use electrosurgical instruments, carrying one or more electrodes, one or more sonotrodes, one or more antennas, or a combination thereof, and electrosurgical generators for providing electrosurgical therapy signals to the electrosurgical instruments.

Since the early development of electrosurgery, both electrosurgical instruments and electrosurgical generators have been improved to provide better results. While, at the onset of electrosurgery, a small number of different electrosurgical instruments have been used for a variety of procedures, more and more specialized electrosurgical instruments have been developed, which are optimized for performing specific procedures with high efficiency. In parallel, waveforms of electrosurgical therapy signals have been further developed for optimal driving of such specialised electrosurgical instruments.

As a consequence, modern electrosurgical generators are able to provide a variety of electrosurgical therapy signals which are not necessarily compatible with every available electrosurgical instrument. For avoiding functional or safety issues manufacturers of electrosurgical generators have developed a number of proprietary interfaces, to which only certified electrosurgical instruments can be connected. Such interfaces are usually provided with means for identifying an electrosurgical instrument, and a processor controlling the electrosurgical generator is configured to only enable electrosurgical therapy signals compatible with the respective electrosurgical instrument.

To also enable use of other electrosurgical instruments, electrosurgical generators may also provide non-proprietary interfaces. As such non-proprietary interfaces usually do not include means for identifying an electrosurgical instrument connected thereto, it is not possible to determine electrosurgical therapy signals compatible with an electrosurgical instrument connected to the non-proprietary interface. The processor controlling the electrosurgical generator may then be configured to allow only output of a limited variety of electrosurgical therapy signals, which do not pose a risk of functional or safety issues. Therefore, only a small number of standard electrosurgical therapy signals may be available. If, on the other hand, the processor is configured to allow selection of a wider variety of electrosurgical therapy signals to be provided through the non-proprietary interface, it is up to the user to determine compatibility with the electrosurgical instrument. Such determination may be prone to human error.

It would be desirable to provide an electrosurgical generator with improved functionality. It would further be desirably to provide an electrosurgical generator less prone to human error. It would also be desirable to provide an electrosurgical generator offering a broader range of electrosurgical therapy signals through a non-proprietary interface.

### SUMMARY OF THE DISCLOSURE

The present disclosure provides an electrosurgical generator, comprising: at least one interface for connecting an electrosurgical instrument to the electrosurgical generator; an electrosurgical signal generation unit for supplying an electrosurgical signal to an electrosurgical instrument connected to the electrosurgical generator; a processor configured to control the electrosurgical signal generation unit; and an instrument detection unit configured to detect the type of an electrosurgical instrument connected to the electrosurgical generator; wherein the instrument detection unit comprises a camera and an image processor, the camera being configured to acquire one or more images of an electrosurgical instrument connected to the electrosurgical generator, and the image processor being configured to analyse the one or more images to detect the type of the electrosurgical instrument; and wherein the processor is configured to control the electrosurgical signal generation unit depending on the detected type of the electrosurgical instrument.

The electrosurgical generator according to the present disclosure may not need to rely on proprietary mechanisms for instrument identification, and may therefore be capable of providing more versatile electrosurgical therapy signals through a non-proprietary interface.

The camera may be configured to acquire one or more 3D images of the electrosurgical instrument, so that the electrosurgical instrument can be detected with more precision. The camera may be a time-of-flight (TOF) camera.

The image processor may be configured to apply an instrument recognition algorithm on the one or more images acquired by the camera. The instrument recognition algorithm may comprise an object separation step. The instrument recognition algorithm may comprise a feature extraction step.

The electrosurgical generator may comprise a database. The processor may be configured to select a database entry from the database using one or more features returned by the feature extraction step, and to read one or more parameters of an electrosurgical therapy signal from the selected database entry. The instrument recognition may use artificial intelligence (AI) or machine learning (ML).

The present disclosure further provides a method of operating an electrosurgical generator, with the steps: connecting an electrosurgical instrument to the electrosurgical generator; acquiring, through a camera of the electrosurgical generator, at least one image of the electrosurgical instrument; analysing, through an image processor, the one or more images; detecting, through the image processor, the type of the electrosurgical instrument; and controlling, through a processor, an electrosurgical signal generation unit depending on the detected type of the electrosurgical instrument. Analysing the one or more images may include applying an instrument recognition algorithm. The instrument recognition algorithm may include an object separation step. The instrument recognition algorithm may comprise a feature extraction step.

The method may further comprise selecting a database entry from a database using one or more features returned by the feature extraction step, and reading one or more parameters of an electrosurgical therapy signal from the selected database entry. Detecting the type of the electrosurgical instrument may include using AI or ML.

### BRIEF DESCRIPTION OF THE DRAWINGS

The subject of this disclosure is hereinafter further explained at hand of exemplary drawings, whereas the embodiments shown in the drawings and described herein are provided only for the purpose of better understanding, without limiting the scope of protection sought. The figures show:

FIG. 1: an electrosurgical system;

FIG. 2: an electrosurgical generator;

FIG. 3: an illustration of an object separation step;

FIG. 4: an AU/ML system.

### DETAILED DESCRIPTION

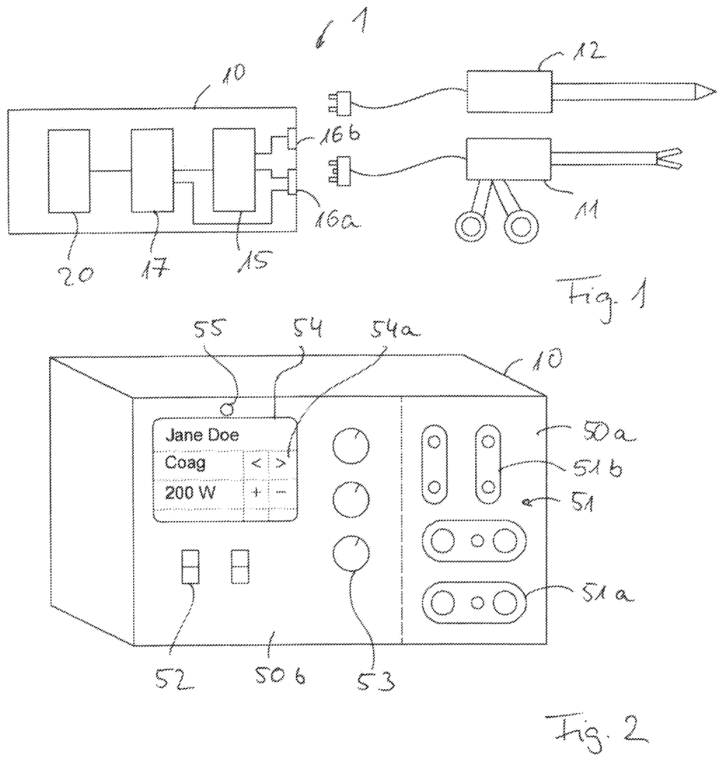

FIG. 1 shows an electrosurgical system 1 with an electrosurgical generator 10 and electrosurgical instruments 11, 12. The electrosurgical generator 10 comprises an electrosurgical signal generation unit 15, which is configured to provide one or more electrosurgical therapy signals to the electrosurgical instruments 11, 12. The electrosurgical instruments 11, 12 can be connected to the electrosurgical generator 10 and the electrosurgical signal generation unit 15 through instrument interfaces 16a, 16b. The electrosurgical generator 10 further comprises a control unit 17 and a user interface unit 20.

The electrosurgical signal generation unit 15 comprises circuitry for generating electrosurgical therapy signals. Such circuitry is generally known to the person skilled in the art, and may include an electronic oscillator for providing a radio-frequency alternating current signal. The electrosurgical signal generation unit 15 may further comprise control circuitry for maintaining voltage, current, and/or power of the alternating current signal at a desired value. The electrosurgical signal generation unit 15 may further comprise signal shaping circuitry for providing the alternating current signal with a desired waveform like sine-wave, square wave, sawtooth wave, or the like.

The electrosurgical signal generation unit 15 is configured to provide sophisticated electrosurgical therapy signals to the electrosurgical instrument 11 through the instrument interface 16a. The electrosurgical signal generation unit 15 is further configured to provide basic electrosurgical therapy signals to the electrosurgical instrument 12 through the instrument interface 16b.

Depending on the desired tissue effect, the electrosurgical signal generation unit 15 may control various parameters of the electrosurgical therapy signals like voltage, current, power, waveform, frequency, pulse-pause ratio, and the like. The electrosurgical signal generation unit 15 may further be configured to monitor the reaction of tissue under treatment by measuring tissue impedance, tissue temperature, tissue moisture, or the like. Such measurement may be performed through dedicated sensors associated with the electrosurgical instrument 11, and/or through indirect measurement based on electrical characteristics of the electrosurgical therapy signal.

Instrument interface 16a is a proprietary instrument interface including dedicated instrument identification features (not shown). Instrument identification features may employ wired or wireless technologies, and are generally known in the art. Such instrument identification features may include, but are not limited to, resistive or reactive electrical elements, memory devices like EEPROM chips. RFID tags, or a combination thereof. Instrument interface 16b is a non-proprietary instrument interface not including any instrument identification features.

The control unit 17 is configured to control operation of the electrosurgical function unit 15. For this purpose, the control unit 17 is configured to receive instrument identification data of the electrosurgical instrument 11 through the instrument identification features of instrument interface 16a. Based on the instrument identification data, the control unit 17 may determine 1s parameters of the electrosurgical therapy signal which are compatible with the electrosurgical instrument 11. The control unit 17 may communicate information regarding such compatible parameters of the electrosurgical therapy signal to the electrosurgical signal generation unit 15.

For the electrosurgical instrument 12, which is connected to the electrosurgical generator 10 through the non-proprietary instrument interface 16b, instrument identification data is not directly available. For still determining compatible parameters of an electrosurgical therapy signal, the electrosurgical generator 10 comprises an instrument detection unit 60 (not shown in FIG. 1), which is explained in more detail further below.

The control unit 17 may further communicate activation/deactivation commands to the electrosurgical function unit 15 to activate or deactivate output of the electrosurgical therapy signal. The electrosurgical function unit 15 may communicate status information and tissue reaction information to the control unit 17.

The control unit 17 may include a processor, memory, and associated hardware known from standard computer technology. The control unit 17 may include program code information stored on the memory for causing the processor to perform various activities of the control unit 17 when executed by the processor. The program code information may include a standard operating system like Windows, MAC-OS, android, Linux, or the like, and/or a proprietary operating system provided by the manufacturer of the electrosurgical generator 10. Such standard computer hardware and operating systems are known to a user and need not be described in detail, here.

The user interface unit 20 is configured to receive status information data from the control unit 17 and to output that status information data to a user, and to allow input of user input data from a user and to communicate that user input data to the control unit 17. The user interface unit 20 may comprise an output device like an electronic display, and one or more input devices like buttons, switches, or knobs. The user interface unit 20 may comprise a combined input/output device like touchscreen. The user interface unit 20 may be integrated into a housing of the electrosurgical generator 10. Some or all components of the user interface unit may be located outside of the housing of the electrosurgical generator 10. Such components may include one or more foot switches (not shown). The user interface unit 20 may comprise data processing hardware separate from the control unit 17, like a processor, memory, and the like. The user interface unit 20 may share some or all data processing hardware with the control unit 17.

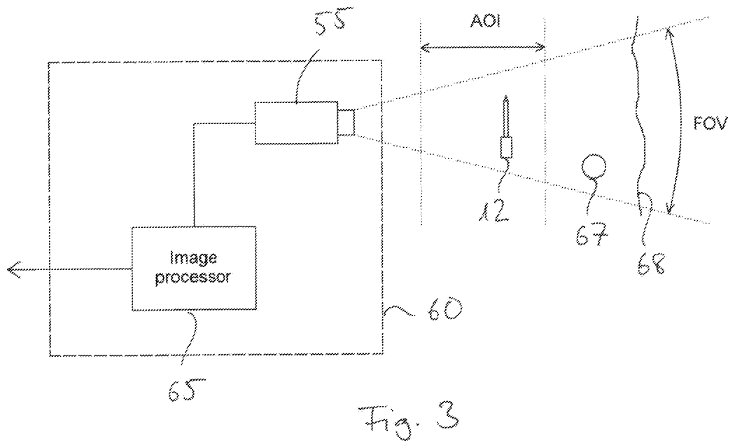

FIG. 2 shows a simplified isometric view of the electrosurgical generator 10. A front panel 50 of the electrosurgical generator 10 includes a connection section 50a and a user interface section 50b.

In the connection section 50a, a plurality of connecting elements 51 are provided, which allow connection of various electrosurgical instruments. The connection section 50a is associated with the electrosurgical signal generation unit 15 of the electrosurgical generator 10. The connecting elements 51 include proprietary connecting elements 51a, corresponding to proprietary instrument interface 16a, and non-proprietary connecting elements 51b, corresponding to non-proprietary instrument interface 16b.

In the user interface section 50b, a plurality of switches 52 and knobs 53 are provided, which allow input of user input data through operation of the switches 52 and/or knobs 53. A display element 54 is provided for outputting of status data. In the shown example, the status data includes a patient name, a selected tissue effect, and a selected output power of an electrosurgical therapy signal. The display element 54 may be a touchscreen, allowing input of further user input data though activation of interactive display elements like “left”/“right” buttons 54a for selecting different tissue effects, or “+”/“−” buttons 54b for increasing or decreasing the selected output power. The user interface section 50b further includes a camera 55, which will be described in more detail below. The user interface section 50b is associated with the user interface unit 20 of the electrosurgical generator 10.

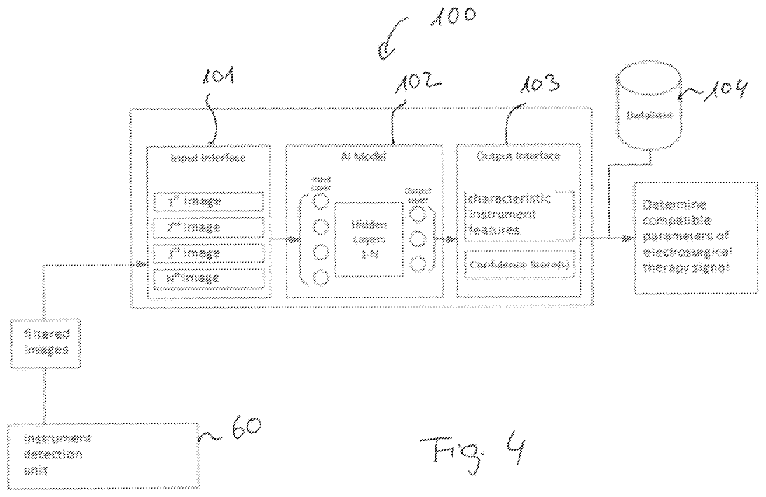

The camera 55 is configured to acquire one or more images of electrosurgical instruments connected to the non-proprietary connecting elements 51b, like electrosurgical instrument 12. In the present example, the camera 55 is a time-of-flight (TOF) camera for acquiring 3D images of the electrosurgical instrument. The electrosurgical generator 10 further comprises an image processor 65 (not shown in FIG. 2) for analysing images acquired by the camera 55. The image processor 65 may be or include a separate processor like a graphical processing unit (GPU), but is not limited thereto. The image processor 65 may likewise be implemented by software executed by one or more processors of the user interface unit 20 or the control unit 17. The camera 55 and the image processor 65 form an instrument detection unit 60 (see FIG. 3).

When an electrosurgical instrument like electrosurgical instrument 12 is connected to one of the non-proprietary connecting elements 51b, the control unit 17 may activate the instrument detection unit 60 in order to identify the type of the electrosurgical instrument. The control unit 17 may further control the user interface unit 20 to display information on the display element 54 prompting a user to place the electrosurgical instrument in the field of view (FOV) of the camera 55.

Upon activation of the instrument detection unit, the camera 55 acquires one or more images, e g. 3D images, of the electrosurgical instrument, and the image processor applies an instrument recognition algorithm on the images acquired by the camera 55.

In some embodiments, a user may be prompted to present the electrosurgical instrument in the field of view of the camera 55 in different orientations, e.g. through rotation of the electrosurgical instrument. A user may further or alternatively be prompted to present the electrosurgical instrument in different operational conditions, e g. with opened and closed jaws, extended and retracted cutting blade, or the like.

In a first step of the instrument recognition algorithm, the image processor may apply an object separation step, as illustrated in FIG. 3.

FIG. 3 shows the instrument detection unit 60 comprising the camera 55 and the image processor 65. The camera 55 is configured to acquire a 3D image of the field of view (FOV). The image comprises a plurality of pixels, e g. 10,000 pixels, each having brightness information, color information (optional), and distance information, wherein the distance information indicated the distance of an object represented by the respective pixel and the camera 55.

The image processor 65 uses the distance information of each pixel for filtering out only pixels representing an object within a certain distance range or area of interest (AOI) from the camera 55. In the example shown in FIG. 3, the filtered image will only show the electrosurgical instrument 12, but not a foreign object 67 or a background 68, which are outside of the area of interest.

The instrument detection unit 60 may be configured to acquire a series of images through the camera 55, and apply the object separation step to each image of the series of images, to obtain a series of filtered images.

After the object separation step, the instrument recognition algorithm may include a feature extraction step. In the feature extraction step, the image processor 65 may analyse the filtered image or the series of filtered images to identify certain characteristic features of the electrosurgical instrument. Such characteristic features may include, but are not limited to:
Handle type (pencil, pistol type, inline type, forceps type, or the like);
Shaft size (none, shaft diameter, shaft length, or the like);
End effector type (fixed, scissors, jaws, or the like);
Electrodes (number, shape, size, or the like).

Besides geometrical features of the electrosurgical instrument, the image processor may also analyse visual identification features which are applied to the electrosurgical instrument without changing the shape thereof, like printed labels, barcodes, QR-codes, or the like, if such features are present with sufficient quality in the filtered image or the series of filtered images. However, as the typical environment in the field may not be optimised for machine-vision applications, such visual identification features are preferably not relied upon as only identification features.

In the feature extraction step, the image processor 65 may employ an artificial intelligence (AI) or machine learning (ML) model. An example of such AI/ML model is explained below with regard to FIG. 4.

FIG. 4 shows a schematic diagram of an exemplary computer-based AI/ML system 100 that is configured to determine characteristic features of an electrosurgical instrument based on filtered input images. In various embodiments, the AI/ML system 100 includes an input interface 101 through which filtered images of an electrosurgical instrument are provided as input features to an artificial intelligence (AI) model 102, and a processor which performs an inference operation in which the filtered images are applied to the AI model to generate a list of characteristic features. The processor may be the image processor 65, or a processor of the control unit 17.

In some embodiments, the input interface 101 may be a direct data link between the A/ML system 100 and the image processor 65 that generates the filtered images. For example, the input interface 101 may transmit the filtered images directly to the AI/ML model 102 during execution of the instrument detection algorithm.

Based on one or more of the filtered images, the processor performs an inference operation using the AI model 102 to generate a list of characteristic instrument features of the electrosurgical instrument. For example, input interface 101 may deliver the filtered images into an input layer of the AI model 102 which propagates these input features through the AI model to an output layer. The AI model 102 can provide a computer system the ability to perform tasks, without explicitly being programmed, by making inferences based on patterns found in the analysis of data. AI model explores the study and construction of algorithms (e.g., machine-learning algorithms) that may learn from existing data and make predictions about new data. Such algorithms operate by building an AI model 102 from example training data in order to make data-driven predictions or decisions expressed as outputs or assessments.

There are two common modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled, and allowing the algorithm to act on that information without guidance. Unsupervised ML is useful in exploratory analysis because it can automatically identify structure in data.

Common tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR). Naive-Bayes, Random Forest (RF), neural networks (NN), deep neural networks (DNN), matrix factorization, and Support Vector Machines (SVM).

Some common tasks for unsupervised ML include clustering, representation learning, and density estimation. Some examples of commonly used unsupervised-ML algorithms are K-means clustering, principal component analysis, and autoencoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In some examples, the AI model may be trained continuously or periodically prior to performance of the inference operation by the processor Then, during the inference operation, the input features provided to the AI model may be propagated from an input layer, through one or more hidden layers, and ultimately to an output layer that corresponds to the characteristic features of the electrosurgical instrument. The characteristic features are then transferred to an output interface 103.

During and/or subsequent to the inference operation, the characteristic features of the electrosurgical instrument may be communicated to the control unit 17. The control unit 17 may then access a database 104 for obtaining compatible parameters of an electrosurgical therapy signal.

Training of the AI/ML system 100 may involve supervised machine learning, wherein a plurality of known electrosurgical instruments are used. Of such known electrosurgical instruments, a number of filtered images will be produced and used as training input data, and a list of known characteristic features of such electrosurgical instruments will be used as training output data.

In some embodiments, the instrument detection algorithm may not include a feature extraction step, but may use an AI/ML system to directly infer compatible parameters for an electrosurgical therapy signal from the filtered images provided to the AI model. In such embodiments, the AI/ML system may also be trained by unsupervised learning, federated learning, or a combination thereof. Here, the instrument detection unit 60 may acquire a number of filtered images of an electrosurgical instrument, and a user of the electrosurgical generator 10 may be requested to input desired parameters of the electrosurgical therapy signal though the user interface unit 20.

The electrosurgical generator 10 may then communicate the filtered images and the parameters input by the user to a centralized server, which can be assessed by a plurality of electrosurgical generators. Together with the filtered images and the parameters, the electrosurgical generator may also communicate information regarding the result of a procedure to the centralized server, e.g. a binary information if the procedure was successful or not.

The centralized server may then use the information received from the plurality of electrosurgical generators for training the AI model, so that the AI model may afterwards infer ranges of compatible parameters for an electrosurgical therapy signal from filtered images of an electrosurgical instrument. The weights table of such trained AI model may afterwards be communicated to a plurality of electrosurgical generators connected to the centralized server.

The instrument recognition algorithm may be designed to return fixed values for relevant parameters of an electrosurgical therapy signal compatible with the recognized electrosurgical instrument. In some embodiments, the instrument recognition algorithm may be designed to return allowed ranges for parameters of the electrosurgical therapy signal. In such embodiments, the control unit 17 may communicate allowable ranges to the user interface device 20, and a user of the electrosurgical generator 10 may input the parameters of the electrosurgical therapy signal within the so determined ranges.

In some embodiments, the control unit 17 may be configured to obtain additional information regarding a recognized electrosurgical instrument from the database 104. Such information may include instructions or recommendations for using the respective electrosurgical instrument. The additional information may be presented to a user of the electrosurgical generator through the user interface unit 20.

1. An electrosurgical generator, comprising:
at least one interface for connecting an electrosurgical instrument to the electrosurgical generator;
an electrosurgical signal generation unit for supplying an electrosurgical signal to an electrosurgical instrument connected to the electrosurgical generator;
a processor configured to control the electrosurgical signal generation unit; and
an instrument detection unit configured to detect the type of an electrosurgical instrument connected to the electrosurgical generator;
wherein the instrument detection unit comprises a camera and an image processor, the camera being configured to acquire one or more images of an electrosurgical instrument connected to the electrosurgical generator, and the image processor being configured to analyse the one or more images to detect the type of the electrosurgical instrument; and
wherein the processor is configured to control the electrosurgical signal generation unit depending on the detected type of the electrosurgical instrument.

2. The electrosurgical generator of claim 1, wherein the camera is configured to acquire one or more 3D images of the electrosurgical instrument.

3. The electrosurgical generator of claim 2, wherein the camera is a time-of-flight (TOF) camera.

4. The electrosurgical generator of claim 1, wherein the image processor is configured to apply an instrument recognition algorithm on the one or more images acquired by the camera.

5. The electrosurgical generator of claim 4, wherein the instrument recognition algorithm comprises an object separation step.

6. The electrosurgical generator of claim 4, wherein the instrument recognition algorithm comprises a feature extraction step.

7. The electrosurgical generator of claim 1, further comprising a database.

8. The electrosurgical generator of claim 6, wherein the processor is configured to select a database entry from the database using one or more features returned by the feature extraction step, and to read one or more parameters of an electrosurgical therapy signal from the selected database entry.

9. The electrosurgical generator of claim 4, wherein the instrument recognition algorithm uses artificial intelligence (AI) or machine learning (ML).

10. A method of operating an electrosurgical generator according to claim 1, with the steps:
connecting an electrosurgical instrument to the electrosurgical generator;
acquiring, through the camera of the electrosurgical generator, one or more images of the electrosurgical instrument;
analysing, through the image processor, the one or more images;
detecting, through the image processor, the type of the electrosurgical instrument; and
controlling, through the processor, the electrosurgical signal generation unit depending on the detected type of the electrosurgical instrument.

11. The method of claim 10, wherein the acquiring of one or more images of the electrosurgical instrument includes acquiring one or more 3D images of the electrosurgical instrument.

12. The method of claim 10, wherein analysing the one or more images includes applying an instrument recognition algorithm.

13. The method of claim 12, wherein the instrument recognition algorithm includes an object separation step.

14. The method of claim 12, wherein the instrument recognition algorithm comprises a feature extraction step.

15. The method of claim 14, further comprising:
selecting a database entry from a database using one or more features returned by the feature extraction step, and
reading one or more parameters of an electrosurgical therapy signal from the selected database entry.

16. The method of claim 12, wherein detecting the type of the electrosurgical instrument includes using AI or ML.
